(19) United States
(12) Patent Application Publication
(10) Pub. No.: US 2005/0004479 A1
(43) Pub. Date: Jan. 6, 2005

(54) Locating features in a photoplethysmograph signal

(76) Inventors: Neil William Townsend, Montargis (FR); Richard Bartholomew Germuska, Oxford (GB)

Correspondence Address: Nathan M Rau;Westman Champlin & Kelly, Internation Ecntre Suite 1600, 900 Second Avenue South, MInneapolis, MN, 55402-3319, US

(21) Appl. No.: 10/490,545

(22) PCT Filed: Sep. 24, 2002
(86) PCT No.: PCT/GB02/04314

(30) Foreign Application Priority Data
Sep. 28, 2001 (GB) 0123395.6

(51) Int. Cl. A61B 5/02

(52) U.S. Cl. 600/500

(57) ABSTRACT

A method and apparatus for locating a feature in a photoplethysmograph or blood pressure signal, comprising a series of signal complexes each having a principal peak (or equivalent trough), is disclosed. The signal is processed to identify a reference point on the upslope of a principal peak. The signal is then searched for the feature in the vicinity of the reference point.

The present invention relates to methods and apparatus for locating features in a photoplethysmograph signal, a blood pressure signal or other similar signal, and in particular, but not exclusively, to the locating of principal peaks or equivalent troughs in an optical transmission, absorption or reflectance signal obtained using a pulse oximeter photoplethysmograph.

Photoplethysmography is a technique used to detect changes in blood perfusion of limbs and tissues, typically by transmitting light through the an ear lobe or finger tip. As arterial pulsations enter the capillary bed, changes in the volume of the blood vessels or characteristics of the blood itself modify the optical properties of the capillary bed.

Pulse oximetry has become a standard means of monitoring arterial oxygen saturation in a noninvasive and continuous manner. Pulse oximeters use photoplethysmography to measure the transmission of two wavelengths of light through blood which absorbs different amounts of light at the two wavelengths depending on the concentration of oxyhemoglobin and deoxygenated hemoglobin. This transmission of light can be modelled using the Beers-Lambert law, and the concentration of each substance arrived at. This allows calculation of the arterial oxygen saturation (SaO₂) of the blood which is given by

$${SaO}_{2} = \frac{{C}_{OX}}{{C}_{OX} + {C}_{DOX}} \qquad ( 1 )$$

where C_{OX} and C_{DOX} are the concentrations of oxyhemoglobin and deoxygenated hemoglobin respectively.

Photoplethysmograph signals, in particular optical transmission or reflectance signals used to derive SaO₂, can generally be divided into two components:
An AC component which is due to the absorption of light in pulsatile arterial blood volume
A DC component caused by the absorption produced by nonpulsatile arterial blood, venous and capillary blood and tissue absorption.

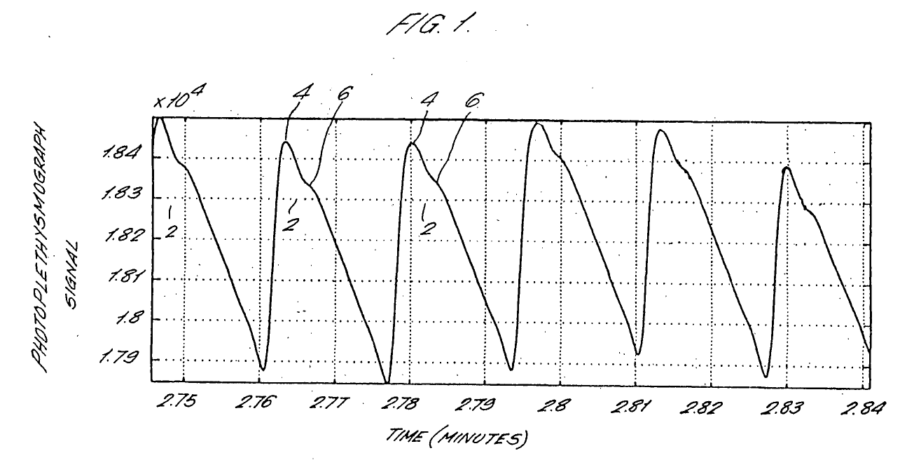

A typical signal from a pulse oximeter photoplethysmograph is shown in FIG. 1. The signal comprises a number of signal complexes 2. The complexes recur at the same rate as the patient's heartbeat. Each complex comprises a principal peak 4 and, in the signal of FIG. 1A, a shoulder 6 following shortly after the principal peak.

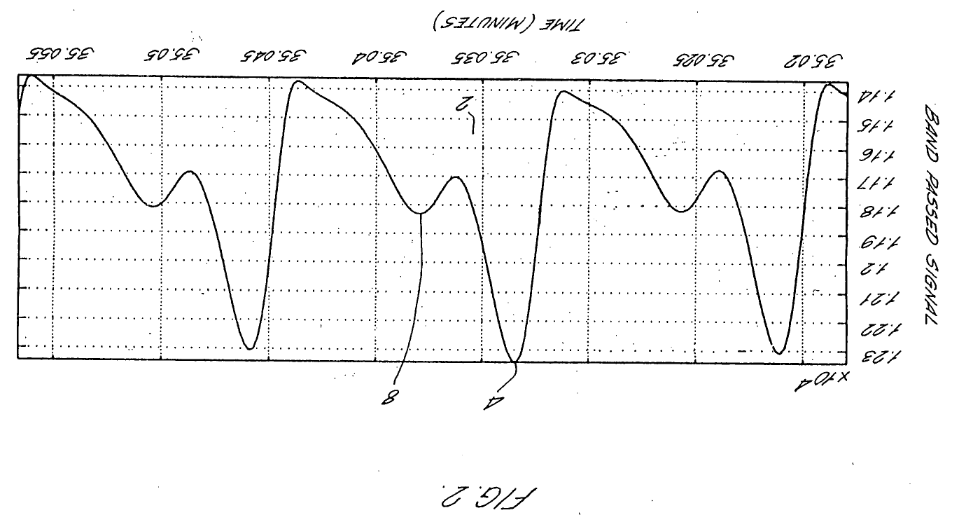

Another typical photoplethysmograph signal is shown in FIG. 2. The shoulder 6 of FIG. 1 has been replaced by a distinct secondary peak called a dichotic notch 8, but the principal peaks are still clear.

Automatic and accurate detection of each principal peak in the AC component of a photoplethysmograph signal would be of considerable use in a number of areas, including:
The accurate determination of pulse rate, which is represented by the time interval between successive principal peaks;
The calculation of pulse transit time (PTT), which may be represented by the time interval between the R-peak recorded by an electrocardiograph heart monitor and the subsequent principal peak detected by the photoplethysmograph; and
The determination of the beat-to-beat variations in blood pressure from PTT.

The pulse transit time is the time taken for a pressure wave in the bloodstream initiated by a heart beat to travel between two locations. The start point may be an R-peak recorded by an electrocardiograph or it may be a clearly defined fiducial point detected in a photoplethysmograph or pressure signal. The end point will be a second such clearly defined fiducial point.

The PTT is acknowledged as being of considerable use in the management of obstructive sleep apnoea patients. Furthermore it has been shown that a beat-to-beat blood pressure may be derived from PTT since a principal determinant of speed of an arterial pressure wave (and therefore the PTT) is the degree of stiffness or tension in the arterial walls, which in turn is determined mostly by the blood pressure. The availability of a beat-to-beat blood pressure measure is also useful in the detection and management of patients suffering from pulsus paradox.

The majority of photoplethysmograph devices currently available rely on simple thresholding or peak detection algorithms to find the principal peaks in a detected signal. These methods are unreliable when the detected signal is less than ideal. Particular problems may be encountered when the baseline of the AC signal component wanders or jumps, when the signal exhibits a marked dichotic notch, and during the occurrence of even mild movement artifacts.

The problem of detecting regular peaks in noisy or complex signals output from particular medical monitoring devices has been addressed from time to time. For example, Pan and Tompkins present a technique for reliably recognising QRS complexes in ECG signals, in IEEE-Transactions on Biomedical Engineering, Vol. BME-32, No. 3, March 1985. However, each signal type from each kind of monitor presents new and different problems, depending on the underlying processes being monitored, the detection methods used and the parameters required from the signal analysis.

The present invention seeks to address problems and disadvantages of the related prior art. Accordingly, the invention provides a method of locating a feature in a digitised photoplethysmograph signal, blood pressure signal or other similar signal, the signal comprising a series of signal complexes each having a principal peak (or equivalent trough), the method comprising the steps of:

processing the signal to identify a reference point on the upslope of a principal peak; and

searching for the feature in the vicinity of the reference point.

The signal may, in particular, be an optical transmission, absorption or reflectance signal obtained using a pulse oximetry photoplethysmograph. Alternatively, the signal may be an intravenous blood pressure signal or signal obtained from a pressure sensor placed on a subject, such as on the subject's arm, foot, finger, wrist or shoulder, for example for measuring a pulse pressure wave resulting from a heartbeat. One signal feature the location of which is of particular interest and utility is the principal peak (which term should be understood to include an equivalent trough, depending on how the signal is presented), of the signal, which generally follows a steep upslope (or equivalent downslope) in the signal. This steep upslope can be used to provide a reference point in each signal complex on the basis of which a search operation can be carried out for the precise location of the principal peak, or of a different feature of the complex. Other features of interest which may be located using the method include the trough between successive signal complexes, the clinically utilised point 25% of the way from the trough to the principal peak, and the dichotic notch, if present.

Preferably, the step of processing includes the step of applying a aradient function to the signal to determine a gradient waveform. The gradient function will typically take the form of a digital filter or discrete differencing function applied to a group of signal points. The application of a gradient function to the data allows the steep upslope to the principal peak of each signal complex to be selected in preference to other parts of the signal which have gradients of lesser magnitude or opposite sign. The steep upslope can be identified as a peak in the gradient waveform, which may then be selected as a reference peak.

Advantageously, a peak enhancement function may be applied to the gradient waveform before the reference peak is selected. A non-linear function such as a square, cubic or exponential function applied to each point of the gradient waveform exaggerates the largest peaks in comparison with smaller peaks, facilitating the process of selecting those peaks in the gradient waveform which correspond to the upslopes of principal peaks in the photoplethysmograph signal.

Preferably, the peak enhancement function retains the sign of each point of the differentiated signal, so that the sense of the gradient of the original signal can be used in determining the reference points, for example by neglecting regions of negative signal gradient.

Preferably, the step of processing further includes the step of discarding a reference peak if it fails to meet a threshold criterion. A convenient way of effecting this step is to discard a reference peak which fails to reach a threshold value. To ensure the method is adaptive to changing signal conditions such as signal complex magnitude, baseline level, movement artifact irregularities and noise, the threshold is preferably adaptive. In particular, the threshold criterion may be calculated using the height of one or more of the preceding reference peaks, for example by taking the average of the heights of two or three preceding peaks and adjusting the average using a preset parameter or function.

The threshold criterion may be further modified if no reference peak meeting the threshold criterion is detected within a predetermined interval. For example, a linear or exponential decay may be applied to the threshold criterion if no peak has been detected within an interval in which at least one signal complex would be expected. This interval may advantageously be set to about two seconds, within which about two patient heartbeats would be expected.

Preferably, the reference point on the upslope of a principal peak is determined from the location of the reference peak, with which it will typically be coincidental.

Advantageously, the step of processing may be carried out on the signal following a step of band-pass filtering of the signal. In this way, interference such as mains power hum, as well as changes in the level of the baseline signal can be removed.

Preferably, the step of searching for the feature comprises the step of scanning the signal in the vicinity of the reference point by applying a predetermined scan criterion to a plurality of points of the signal in the vicinity of the reference point. One way of carrying out this step is to apply a feature detection criterion to each signal point in turn, moving in one or two directions from the reference point, until a point satisfying the feature detection criterion is satisfied. The criterion could be as simple as seeking a signal point having lesser magnitude neighbouring points on both sides, in order to detect a local peak, or could take the form of a more sophisticated convolution function.

Alternatively, the step of searching for the feature may comprise a step of fitting a curve such as a smoothed cubic spline to the signal in the vicinity of the reference point and identifying the feature from a corresponding feature in the fitted curve such as a peak, trough or point of inflection.

Advantageously, the step of searching for the signal peak may be carried out on the signal following band pass filtering of the signal.

The invention may be embodied in apparatus, such as a general purpose computer apparatus, a dedicated photoplethysmograph or another medical apparatus programmed to carry out the steps of the method described above.

The invention may also be embodied in a computer readable data carrier carrying computer program instructions which cause the method to be carried out when executed on a computer.

Preferred embodiments of the invention will now be described, by way of example only, with reference to the accompanying drawings, of which:

FIG. 1 shows a typical signal output from a pulse oximeter photoplethysmograph;

FIG. 2 shows a typical signal output from a pulse oximeter photoplethysmograph, following application of a band pass filter, and exhibiting dichotic notch features;

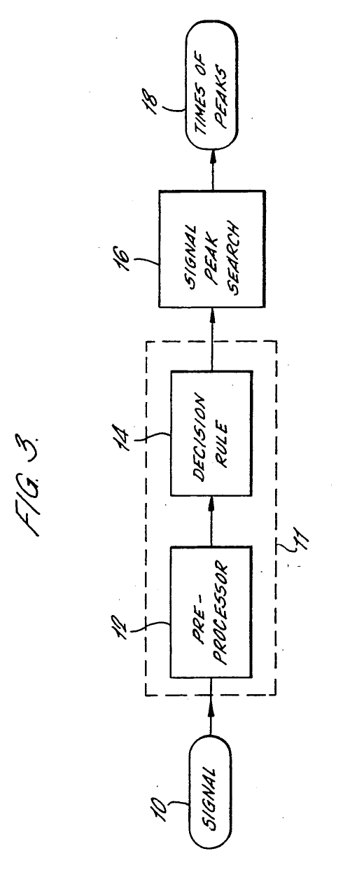

FIG. 3 is a schematic diagram showing principal method steps of preferred embodiments of the invention;

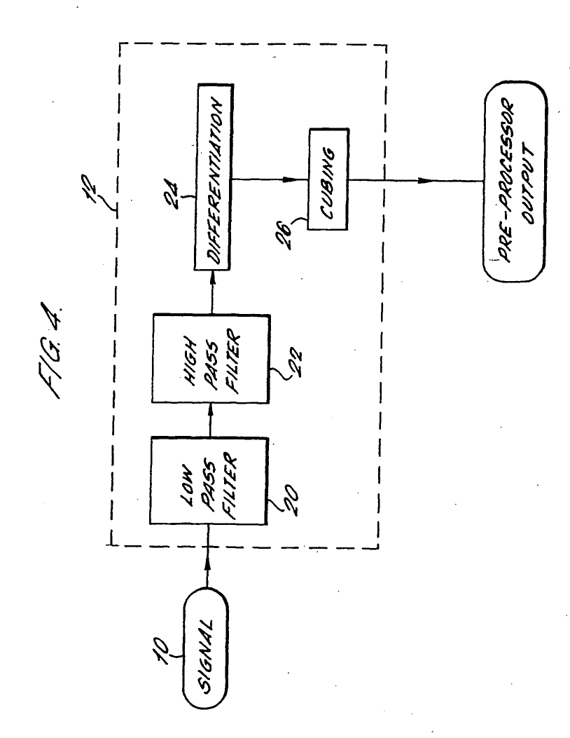

FIG. 4 is a schematic diagram showing elements of the pre-processing step of FIG. 3;

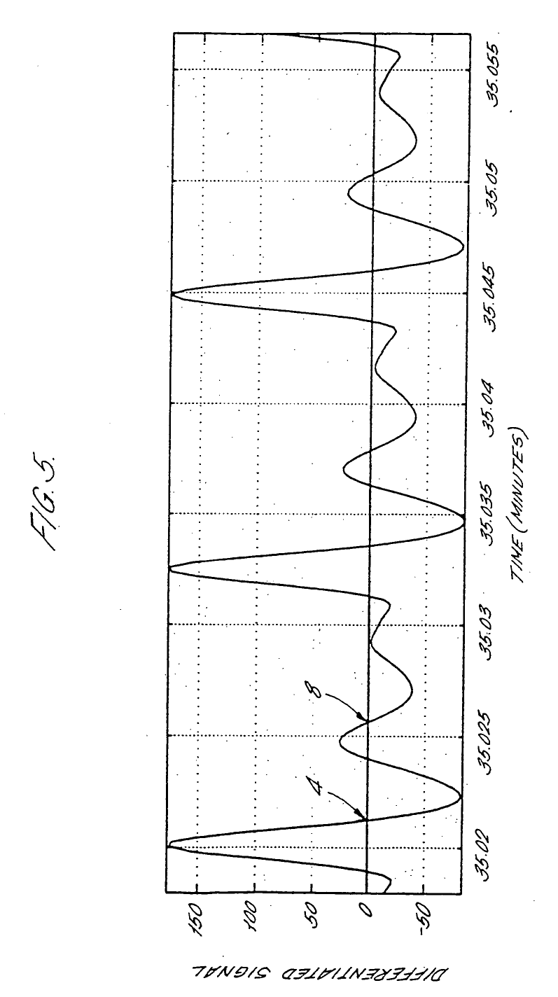

FIG. 5 shows a gradient waveform derived by differentiation of the signal of FIG. 2;

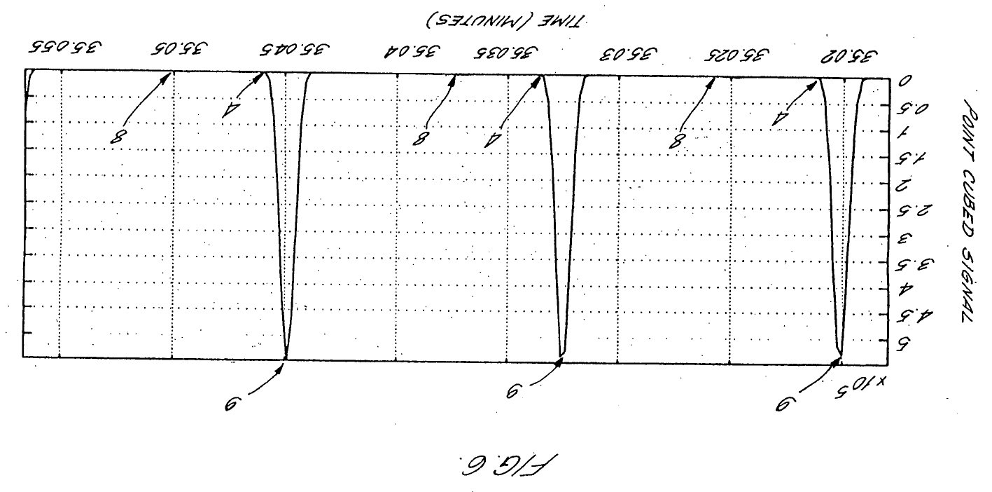

FIG. 6 shows the gradient waveform of FIG. 5 following peak enhancement by application of the cubing function of FIG. 4;

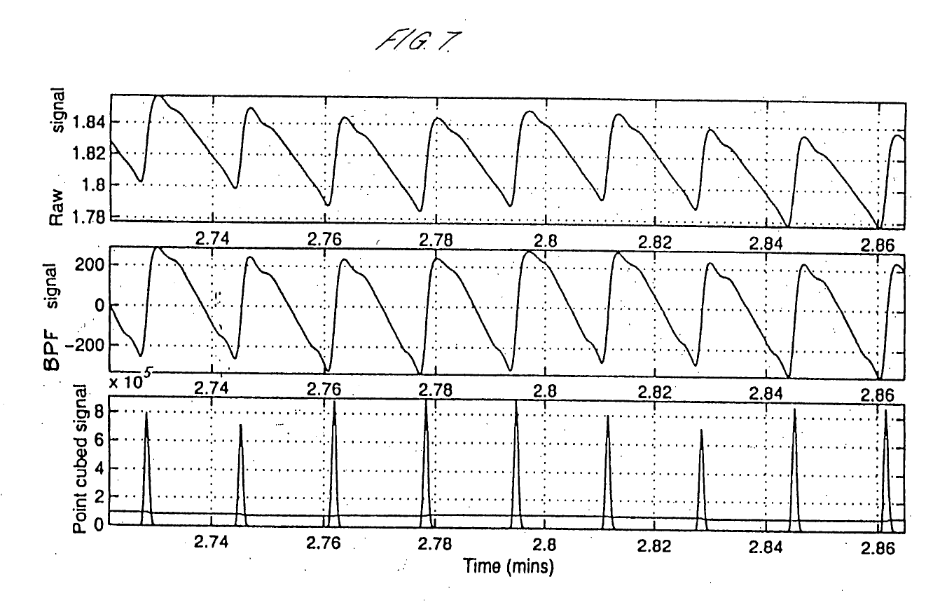

the lower panel of FIG. 7 shows the output from the pre-processor of FIG. 4, corresponding to the input signal shown in the upper panel;

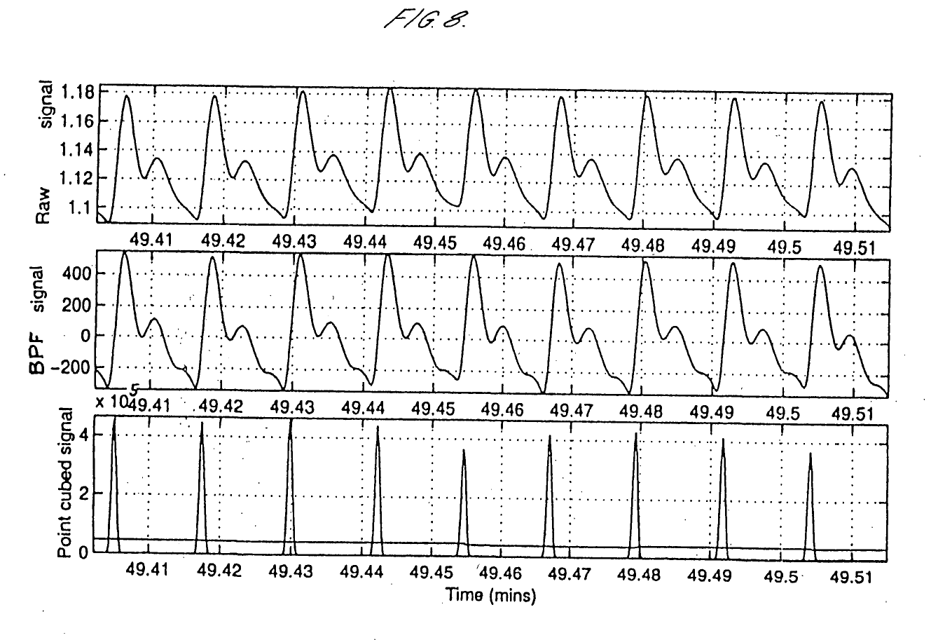

the lower panel of FIG. 8 shows the output from the pre-processor of FIG. 4, corresponding to the input signal shown in the upper panel, which exhibits dichotic notch features;

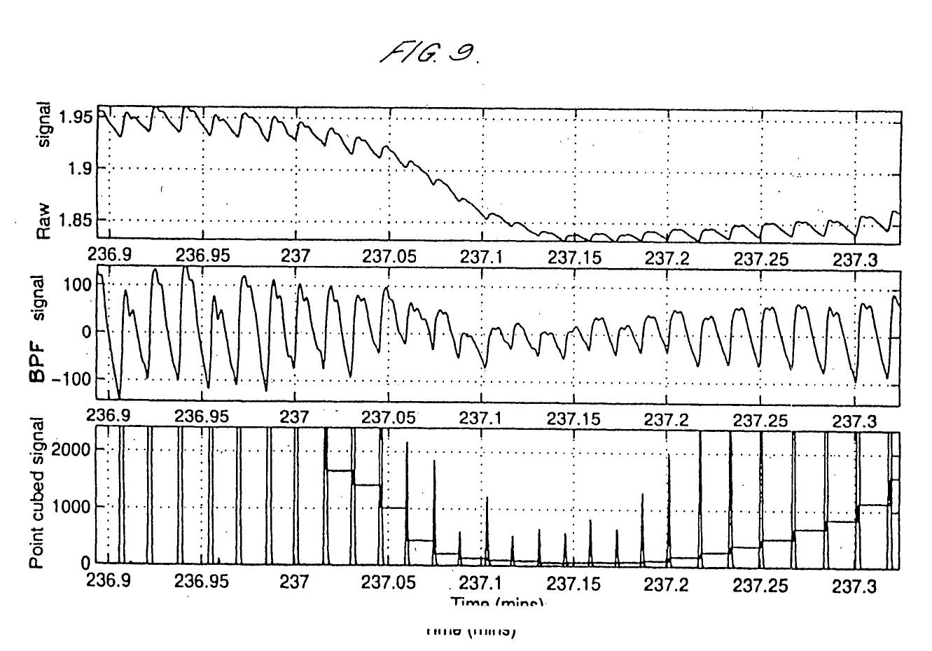

the lower panel of FIG. 9 shows the output from the pre-processor of FIG. 4, corresponding to the input signal shown in the upper panel, which exhibits baseline shift;

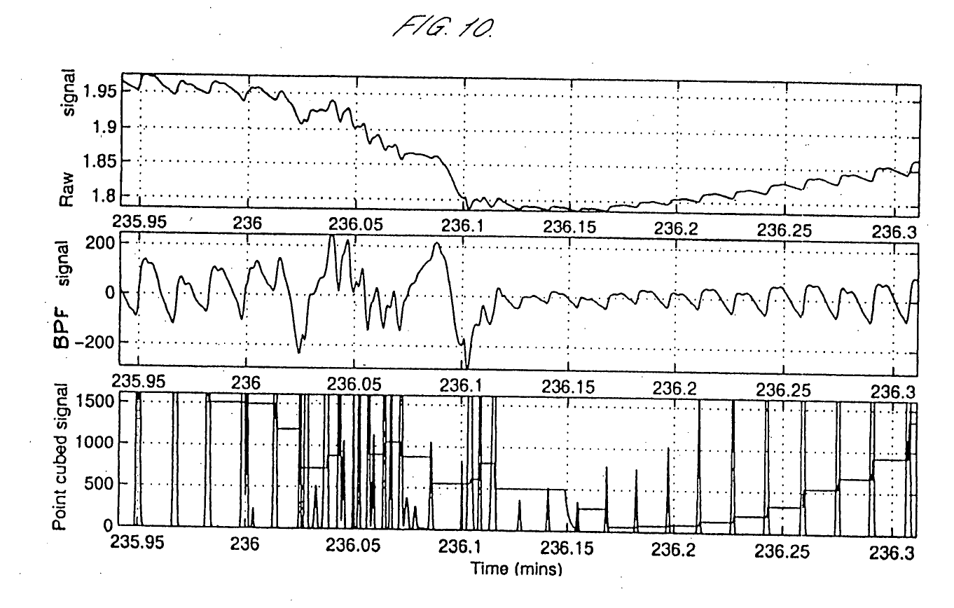

the lower panel of FIG. 10 shows the output from the pre-processor of FIG. 4, corresponding to the input signal shown in the upper panel, which exhibits movement artifact irregularities;

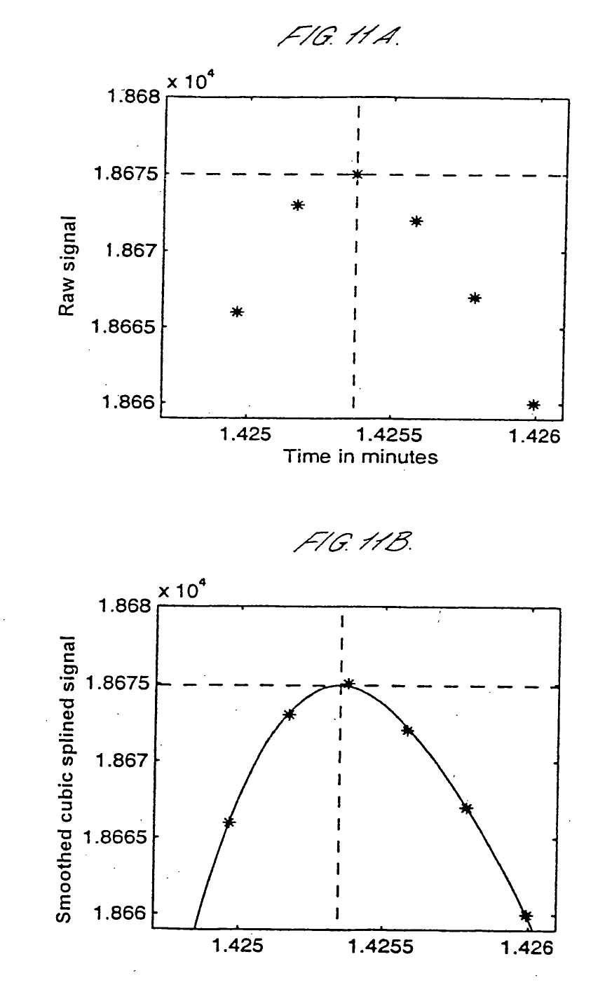

FIG. 11A shows a plot of some raw photoplethysmograph signal data points, with the principal peak identified by a scan forward method identified by crosshairs;

FIG. 11B shows a plot of the same data points as shown in figure 11A, with the principal peak identified by a spline fitting method identified by crosshairs; and

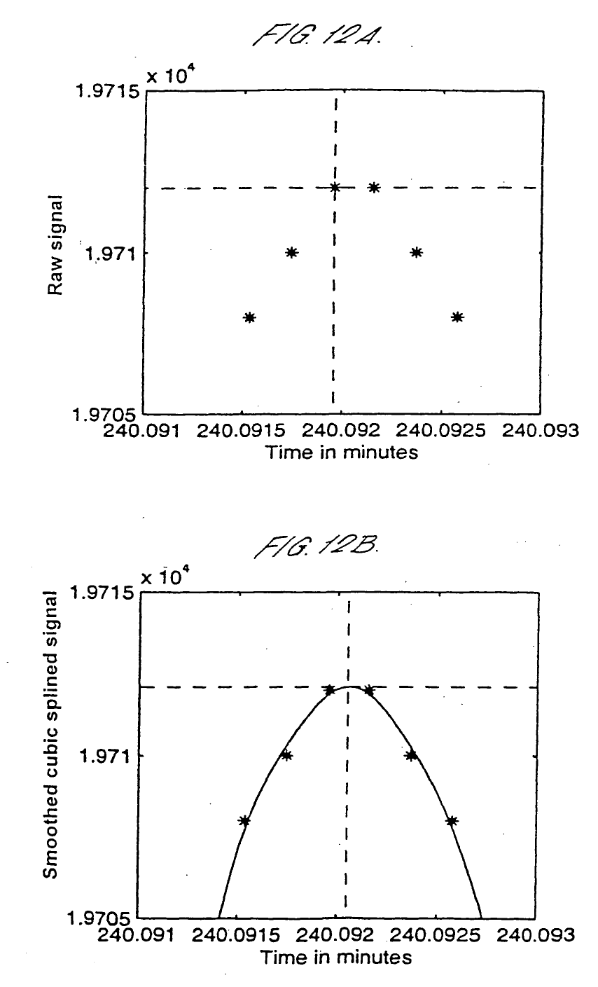

FIGS. 12A and 12B correspond to figures 11A and 11B, but for a different set of raw photoplethysmograph signal data points.

Preferred embodiments of the invention provide methods for detecting principal signal peaks in a photoplethysmograph signal. Such a signal may be obtained, for example, from a Nellcor model MP304 pulse oximeter photoplethysmograph, which includes a filter to eliminate respiratory variation in the AC signal component to the extent that it is found in normal patients. In particular, the embodiments as described here are applied to a signal or signals suitable for deriving a measure of arterial oxygen saturation, or SaO₂. However, the invention is also applicable to other comparable signals derived using photoplethysmography methods, blood pressure measurement methods and the like, and can easily be applied to locate features other than the principal peak of a signal complex. Comparable signals include intravenous blood pressure signals and signals from pressure sensors placed on a subjects body for purposes such as measuring a pulse pressure wave resulting from a heart beat.

FIG. 3 illustrates how the preferred embodiments can be divided into three functional sections or units implemented in hardware, software, or a combination of the two. The signal 10 is first passed to a pre-processor stage 12 which performs linear and non-linear filtering of the signal, and produces a set of well defined pre-processor output signal peaks, each of which corresponds to a signal complex. A decision rule section 14 then operates on the output of the pre-processor 12, and identifies those pre-processor output signal peaks which correspond to principal signal peaks. The centre of each principal signal peak is then located, in stage 16, using one of a number of forward search algorithms that operate with reference to the locations of the peaks of the pre-processor output signal. The preprocessor section 12 and decision rule section 14 may be considered together or combined as a signal processing unit 11.

Pre-processor Process

The steps carried out on the signal 10 by the pre-processor 12 are illustrated in FIG. 4. The signal 10 is first subject to a band pass filter made up of a low pass filter 20 and a high pass filter 22. The low pass filter 20 is an 89 coefficient low-pass equi-ripple FIR filter and the high pass filter is a 309 coefficient high-pass equi-ripple FIR filter. Together they form a 0.8 Hz to 40 Hz band-pass filter with a 40 dB attenuation in the stop-band, designed to remove 50 Hz mains noise (or 60 Hz in some countries) and low frequency baseline shifts which occur due to longer term variations in oxygen saturation caused, for example, by changes in patient breathing rate.

The band-pass filtering process tends to amplify the minor inflexion often found at the end of a signal complex. However, this distortion is not problematic for the process of principal peak detection.

Following band-pass filtering the signal is passed to a numerical differentiation process 24. The difference equation for the numerical differentiation is given by

$$y ( {T}_{n} ) = \frac{2 \times ( {T}_{n} ) + x ( {T}_{n - 1} ) - x ( {T}_{n - 3} ) - 2 \times ( {T}_{n - 4} )}{8} \qquad ( 2 )$$

where x(Tₙ) is the magnitude of the filtered signal at time point Tₙ, and y is the differentiation process output. Various other gradient functions could be used. The effects of the differentiation process 24 on the signal illustrated in FIG. 2 are shown in FIG. 5. It can be seen that the differentiation process 24 highlights those sections of the signal with the largest positive and negative gradients, as expected.

From FIG. 5 it can be seen that the largest positive peaks of the differentiated signal occur at points corresponding to the up-slopes of the principal peak of each signal complex and that the gradient of the upslope to each dichotic notch 8 is of lesser magnitude. The downslope following each principal peak 4 has a large negative gradient, but this is of lesser absolute magnitude than the gradient maximum for the corresponding upslope.

Following differentiation, the signal is passed to a cubing process 26 which arithmetically cubes each point of the signal, and then sets any negative values to zero. By cubing the differentiated signal, the dynamic range is emphasised so as to enhance the gradient peak corresponding to the up-slope of each principal peak relative to the gradient peak corresponding to the up-slope of each dichotic notch. Advantageously, the cube function also retains information regarding the sign of the differentiated signal, so that negative gradients, which are to be neglected, are now set to zero.

The output from the cubing process is illustrated in FIG. 6. The significant peaks correspond to the points of maximum gradient on the up-slopes to the principal peak 4 shown in FIG. 2. The only secondary peaks are those corresponding to the up-slopes of the dichotic notches 8, and these are barely visible.

Decision Rule Process

The signal output from the pre-processor 12 is passed to a decision rule process 14. The decision rule process 14 aims to select those peaks of the pre-processor output signal which correspond to a gradient maximum on the up-slope of a principal peak of a signal complex.

To detect peaks in the pre-processor output signal the decision rule process 14 scans through the signal and identifies peaks using a three-point scheme, although various other schemes could be used. If the second of three adjacent signal points has a value higher than the first and third points then a peak has been identified.

Each peak identified in the pre-processor output signal is tested against an adaptive threshold. Each peak having a signal value greater than the threshold is accepted as an appropriate reference point on the basis of which a search for the adjacent principal peak in the signal can be carried out. Pre-processor output peaks having a signal value lower than the threshold are discarded.

The adaptive threshold is calculated by averaging the values of the pre-processor output signal at each of the two previous identified peaks and multiplying the average by a constant. For the processing of signals similar to those shown in FIGS. 1 and 2 a suitable value for the constant is 0.1.

When the signal peak detection process of a preferred embodiment is applied to a section of a photoplethysmograph signal that is severely corrupted, for example due to physiological movement artifact irregularities, large and irregular peaks can be generated in the pre-processor output signal. These peaks can interfere with the appropriate setting of the adaptive threshold. To ensure recovery of the threshold to an appropriate level, once a clean signal is again provided, an exponential decay is applied to the adaptive threshold if no peak is detected by the decision rule module within a two second interval.

The adaptive thresholding also enables the embodiment to automatically initialise to the scale of a new signal, which depends on what probe is used, coupling to the patient, and the patient themself. It also allows automatic adaption when external conditions such as ambient light levels, patient condition and so on change.

Signal Peak Search Process

Each reference point identified by the decision rule process 14 is passed to the signal peak search process 16, which seeks to identify the precise location of the corresponding principal peak in the subsequent signal. In the preferred embodiments this is carried out either by means of a simple scan forward method or by means of a spline fitting method. Either method can be applied either to the raw signal or to the signal following band pass filtering by filters 20, 22.

In the scan forward method a three point scheme is used to identify as a principal signal complex peak the first signal point which is higher than its neighbours, on scanning forward from a reference point.

In the spline fitting method a preliminary peak is first identified in the signal using the scan forward method. A smoothed cubic spline is then used to provide an interpolation of the signal in the region of the preliminary peak. The region may encompass, for example, 15 signal points before the preliminary peak, the preliminary peak itself, and 15 signal points following the preliminary peak. The peak of the smoothed cubic spline is then identified as a principal signal complex peak.

Smoothed cubic splines, and methods of using such splines to provide a “best fit” to noisy data are discussed in “A practical guide to Splines”, De Boor, Applied Mathematics Sciences Vol. 27, xxiv+329p, Springer V. 1978.

Test Results

The results of testing the described peak detection algorithms on four different classes of pulse oximetry photoplethysmograph signal will be discussed. The four classes are as follows:
1. a signal in which the signal complexes do not exhibit dichotic notch features;
2. a signal in which the signal complexes do exhibit dichotic notch features;
3. a signal with a variable baseline component underlying the signal complexes of interest;
4. a signal exhibiting severe irregularity due to movement artifacts.

Known methods used to identify principal peaks in pulse oximeter photoplethysmograph signals are prone to misidentifying a dichotic notch as the principal peak of a signal complex. Known methods which rely on peak magnitude are also prone to errors when applied to signals with significant baseline shifts. It is also important for a peak detection process to recover after encountering irregular signal sections heavily influenced by movement artifacts.

Each of FIGS. 7 to 10 displays three graphs each having time (in minutes) as the abscissa. In each figure, the upper panel displays a raw photoplethysmograph signal, the middle panel display the signal following band pass filtering as discussed above, and the lower panel displays the corresponding output from the pre-processor 12. A graph showing the level of the adaptive threshold has been superimposed on each lower panel.

FIG. 7 relates to the first class of data mentioned above, the raw signal in the upper panel exhibiting a mild inflection after each principal peak, but not exhibiting any dichotic notch features. The corresponding output from the pre-processor, shown in the bottom panel, is a series of well defined and regular peaks, each peak corresponding to the point of maximum gradient in advance of a principal peak in the raw signal.

FIG. 8 relates to the second class of data mentioned above, the raw signal in the upper panel exhibiting a clear dichotic notch feature in each signal complex. Again, the corresponding output from the pre-processor, shown in the bottom panel, is a series of well defined and regular peaks which are easy for the subsequent decision rule process to identify.

FIG. 9 relates to the third class of data mentioned above, in which the raw signal shown in the upper panel exhibits a significant change in the baseline component underlying the signal complex signal of interest, for example due to a rapid change in the mean oxygen saturation level in a patient being monitored. The baseline component is removed by the band pass filtering, as can be seen in the middle panel, and the peaks in the pre-processor output signal shown in the lower panel are all well defined and all correspond to the upslope of a principal peak of an SaO₂ complex in the raw signal. A graph showing the level of the adaptive threshold subsequently used by the decision rule process to identify which peaks should be discarded has been superimposed on the lower panel. It is clear that the adaptive threshold remains at a suitable level to distinguish relevant peaks despite the large dynamic range of the signal complexes present in the signal.

FIG. 10 relates to the fourth class of data mentioned above, in which the raw signal shown in the upper panel exhibits marked movement artifact irregularity. The regular signal complexes are completely obscured in part of the signal. During the irregular section of the raw signal the pre-processor output signal exhibits many spurious peaks and the adaptive threshold, superimposed on the lower panel, moves erratically. However, when the signal becomes regular again the threshold adapts quickly to fall below the normal pre-processor output peaks which mark the principal peak of each signal complex in the expected manner.

It has been found that the principal peaks in a pulse oximeter photoplethysmograph signal can be identified with reasonable accuracy by both the simple scan forward and more sophisticated smoothed spline fitting methods discussed above. In general, the smoothed spline method appears to perform slightly better, especially on more noisy or less well defined peaks.

FIG. 11A is a graph of some discrete data points from a raw photoplethysmograph signal, in the region of a principal peak of a signal complex. Broken crosshairs identify the principal peak as established using the above described scan forward method. In FIG. 11B the same raw signal data points are shown, but a spline curve established using the smoothed spline fitting method is also shown, with broken crosshairs identifying the principal peak as established from the spline curve.

FIGS. 12A and 12B are equivalent to FIGS. 11A and 11B, but for a different set of raw pulse oximeter photoplethysmograph signal data points. The locations of the principal peak in figures 11A and 11B as established using the scan forward and spline fitting methods are very close together, because a raw data point happens to lie close to the location of the peak established by the spline fitting method. The locations of the principal peak in FIGS. 12A and 12B as established using the scan forward and spline fitting methods are further apart, because the peak established by the spline fitting method lies between two raw data points. In general, the optimum peak location is recovered with better accuracy using the spline fitting method, due to the sampling rate limitations inherent in the scan forward method.

Applying either the scan forward or the smoothed spline fitting method to a band pass filtered signal tends to result in the identified peak being delayed by a few milliseconds relative to the corresponding peak identified using a raw signal. This artifact of the filtering process tends to be more significant when the signal baseline is falling rapidly, and in signal complexes exhibiting a dichotic notch feature.

Although the described embodiment uses a single band pass filter prior to differentiation, other arrangements may be used. It should be noted that a second band pass filter may be used in conjunction with, or instead of, the filter previously described. A signal will be subjected to the second band pass filter subsequent to the differentiation step. The characteristics of the second band pass filter may be similar to those of the first band pass filter. Additionally, as the first and second band pass filters are included to reduce noise, it may not be necessary to include either of the filters. In other words, the signal may be differentiated and then subjected to the cubing process 26 without encountering any filtering. However, as would be appreciated, the noise present in such a system will increase. High pass, low pass or notch filters could be used as well as or instead of band pass filters, to optimise the described arrangements.

1. A method of locating a feature in a digitised photoplethysmograph signal comprising a series of signal complexes each having a principal peak, the method comprising the steps of: processing the signal to identify a reference point on the upslope of a principal peak; and searching for the feature in the vicinity of the reference point:

2. The method of claim 1 wherein the step of processing includes the step of applying a gradient function to the signal to determine a gradient waveform.

3. The method of claim 2 wherein the step of processing further includes the step of detecting a reference peak in the gradient waveform.

4. The method of claim 2 wherein the step of processing further includes the steps of: applying a peak enhancement function to the gradient waveform; and detecting a reference peak in the peak enhanced gradient waveform.

5. The method of claim 4 wherein the peak enhancement function comprises a cube function.

6. The method of claim 3 wherein the step of processing further includes the step of discarding the reference peak if it fails to meet a threshold criterion.

7. The method of claim 6 wherein the threshold criterion is calculated using the size of one or more of the preceding reference peaks.

8. The method of claim 6 wherein the threshold criterion is modified if no reference peak meeting the threshold criterion is detected within a predetermined interval.

9. The method of claim 4 wherein the reference point on the upslope of a principal peak is determined from the location of the reference peak.

10. The method of claim 1 wherein the step of processing includes a preliminary step of applying a band-pass filter to the signal.

11. The method of claim 1 wherein the step of searching for the feature comprises the step of scanning the signal in the vicinity of the reference point by applying a predetermined scan criterion to a plurality of points of the signal in the vicinity of the reference point.

12. The method of claim 1 wherein the step of searching for the feature comprises the step of fitting a curve to the signal in the vicinity of the reference point and identifying the feature from a corresponding feature in the fitted curve.

13. The method of claim 11 wherein the step of searching for the signal peak is carried out on the signal following a step of band pass filtering of the signal.

14. The method of claim 1 wherein the feature is the principal peak of a signal complex.

15. The method of claim 1 further—20 comprising the step of determining a pulse rate from the timings within said signal of a plurality of said features.

16. Apparatus for locating multiple instances of a feature in a digitised photoplethysmograph signal which comprises a series of signal complexes each having a principal peak, the apparatus comprising: a signal processing unit adapted to receive said signal and to identify reference points on the upslopes of said principal peaks; and a search unit adapted to receive said reference points and to search said signal for said feature in the vicinity of each reference point.

17. The apparatus of claim 16 wherein the signal processing unit is adapted to apply a gradient function to said signal to determine a gradient waveform.

18. The apparatus of claim 17 wherein the signal processing unit is further adapted to detect reference peaks in the gradient waveform.

19. The apparatus of claim 17 wherein the signal processing unit is further adapted to apply a peak enhancement function to the gradient waveform and to detect reference peaks in the peak enhanced gradient waveform.

20. The apparatus of claim 19 wherein the peak enhancement function comprises a cube function.

21. The apparatus of claim 18 wherein the signal processing unit is further adapted to discard a reference peak if it fails to meet a threshold criterion.

22. The apparatus of claim 21 wherein the signal processing unit is adapted to calculate a threshold criterion for a particular reference peak using the magnitude of one or more of the preceding reference peaks.

23. The apparatus of claim 21 wherein the signal processing unit is adapted to modify the threshold criterion if no reference peak meeting the criterion is detected within a predetermined interval of the signal.

24. The apparatus of claim 19 wherein the signal processing unit is adapted to determine the reference point on the upslope of each principal peak from the location of a corresponding reference peak.

25. The apparatus of claim 16 further comprising at least one band pass filter arranged to filter the signal either before or after the application of a gradient function to the signal.

26. The apparatus of claim 16 wherein the search unit is adapted to scan the signal in the vicinity of each said reference point by applying a predetermined scan criterion to a plurality of signal points in the vicinity of each said reference point.

27. The apparatus of claim 16 wherein the search unit is adapted to fit a curve to the signal in the vicinity of each said reference point and to identify an instance of the feature from a corresponding feature in the fitted curve.

28. The apparatus of claim 26 further adapted to band pass filter the signal before carrying out the step of scanning or fitting.

29. The apparatus of claim 16 wherein the features for location are the principal peaks of signal complexes.

30. The apparatus of claim 16 further comprising a pulse rate unit adapted to calculate a pulse rate from the locations within said signal of a plurality of said features.

31. A method for locating a feature in a digitised blood pressure signal comprising a series of signal complexes each having a principal peak, the method comprising the steps of: processing the signal to identify a reference point on the upslope of a principal peak; and searching for the feature in the vicinity of the reference point.

32. Apparatus for locating multiple instances of a feature in a digitised blood pressure signal which comprises a series of signal complexes each having a principal peak, the apparatus comprising: a signal processing unit adapted to receive said signal and to identify reference points on the upslopes of said principal peaks; and a search unit adapted to receive said reference points and to search said signal for said feature in the vicinity of each reference point.

33. A computer program product for locating multiple instances of a feature in a digitised photoplethysmograph or blood pressure signal having a series of signal complexes each having a principal peak, the product comprising a computer readable storage medium carrying computer program instructions providing: a signal processing element adapted to receive said signal and to identify reference points on the upslopes of said principal peaks; and a search element adapted to receive said reference points and to search said signal for instances of said feature in the vicinity of each reference point.

34. A photoplethysmograph adapted to carry out the method steps of claim 1.

35. A computer readable data carrier comprising computer program instructions for carrying out the method steps of claim 1 when executed on suitable computer apparatus.

36. A computer readable data carrier comprising computer program code, for locating a feature in a digised blood pressure signal comprising a series of signal complexes each having a principal peak, when executed on a computer, the program code including elements adapted to: process the signal to identify a reference point on the upslope of a principal peak; and search for the feature in the vicinity of the reference point.
